(19) United States
(12) Patent Application Publication
(10) Pub. No.: US 2024/0348675 A1
(43) Pub. Date: Oct. 17, 2024

(54) A VIRTUAL SERVER, MEDICAL SYSTEM, COMPUTER PROGRAM AND METHOD

(71) Applicant: Sony Group Corporation, Tokyo (JP)

(72) Inventor: Bob KONINCKX, Stuttgart (DE)

(73) Assignee: Sony Group Corporation, Tokyo (JP)

(21) Appl. No.: 18/574,760

(22) PCT Filed: Jun. 30, 2022
(86) PCT No.: PCT/EP2022/068211

§ 371 Date: Dec. 28, 2023

(30) Foreign Application Priority Data
Jul. 6, 2021 (EP) 21184003.8

(51) Int. Cl. H04L 65/75; H04L 67/125

(52) U.S. Cl. CPC H04L 65/75; H04L 67/125

(57) ABSTRACT

A virtual server configured to receive and transfer data over an Internet Protocol (IP) network, the virtual server comprising: a medical application configured to process video data relating to a medical procedure to produce an output video stream, the medical application being provided on a Guest Operating System within the virtual server; and an IP converter application sat beneath the Guest Operating System configured to receive the output video stream from the medical application and to convert the at least one video stream into IP packets and to transfer the converted video data over the IP network.

### BACKGROUND

### Field of the Disclosure

The present technique relates to a virtual server, medical system, computer program and method.

### Description of the Related Art

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present technique.

In a medical environment, such as in a hospital with one or more operating rooms, electronic equipment is being used more extensively to improve medical workers' and patients outcomes. For example, the medical solution made by Sony Corporation R exists which allows the planning, recording and sharing of video and still images of a surgical procedure as well as patient related data to be shared amongst clinical staff.

However, such an increase in electronic equipment leads to an increased amount of space being used to house such equipment. This restricts the movement of the surgical team and increases the number of hazards present in the operating room. This also leads to increased amounts of sterilisation and the like. This is undesirable.

Accordingly, there has been an increased use of software in the medical environment which aims to remove as much hardware as possible from the operating room and place as much of the functionality of such hardware in software which would be run on a workstation. In a further step, to reduce the space used by such workstations, the software run on the workstations is being virtualised. In other words, the software from such workstations instead of being run locally in the operating room is instead being run on large servers located in a server room. This virtualisation of medical software, however, is complicated.

More specifically, there are four concerns to be solved when virtualisation of medical software is adopted. Firstly, as such software previously run on the local workstations usually includes video processing which output multiple video streams, it is complicated to virtualize such software as additional hardware is required at the virtual server to provide multiple video outputs. Additionally, in hospital application, the location and the numbers of keyboard and/or mice are concerns. End users want to remotely control a keyboard and/or mouse from other sites in the hospital, and to also reduce the number of keyboard/mouse in order to save space and cost. The first concern is how to treat keyboard, video stream and mouse (namely, KVM) when the virtualisation is adopted.

Secondly, less porting effort for virtualisation is expected. When the software worked on a standalone local workstation is virtualised on large servers located in a server room, some modifications are required such as memory control, disk control, etc in addition to the KVM control, as the software is basically designed for specific hardware, operating system, interface and so on. This modification generally requires development efforts (human resource, development environment, cost, time etc). Moreover, it indirectly connects to the reduction of quality or reliability. Further, it causes the additional maintenance cost after the production launch caused by the modification from the original software which worked on the standalone local workstation. No modification is desired to avoid these concerns.

Thirdly, similarly to the second concern, minimization of the impact to the existing other software is desired. When the software that worked on the standalone local workstation is virtualised on large servers located in a server room, the impact to other software not only working on virtualised workstation but also working on other areas should be minimized.

Lastly, software run on local workstations is often classified as a medical device and is subjected to regulatory approval. Therefore, by simply placing the software on a virtual server will mean that all applications within the virtual server (including its operating system) would need the same regulatory approval to operate by virtue of the presence of the software previously run on the local workstations. When the software which worked on standalone local workstation is virtualised on large servers located in a server room, the clear separation of medical device and non-medical device is desirable for all software used in the hospital in terms of intended use of the software and impact analysis to the effectiveness and safety for medical usage.

It is an aim of the disclosure to address at least one of these issues.

### SUMMARY

According to embodiments of the disclosure, there is provided a virtual server configured to receive and transfer data over an Internet Protocol (IP) network, the virtual server comprising: a medical application configured to process video data relating to a medical procedure to produce an output video stream, the medical application being provided on a Guest Operating System within the virtual server; and an IP converter application sat beneath the Guest Operating System configured to receive the output video stream from the medical application and to convert the at least one video stream into IP packets and to transfer the converted video data over the IP network.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows a medical system 1000 connected to a network:

FIG. 2 shows a server 130 according to embodiments of the present disclosure

FIG. 3 shows a schematic diagram of the application layers in an OR workstation and/or a laboratory workstation:

FIG. 4 shows a schematic diagram of the transmission side of the physical IP Converter 800 A-C (IPC-TX) in FIG. 1:

FIG. 5 shows a medical system 1000′ according to embodiments of the disclosure:

FIG. 6 shows the transmission side of a virtual IP converter 8000 according the embodiments of the disclosure:

FIG. 7A shows a flowchart explaining the operation of the transmission side of the virtual IP Converter with respect to video; and

FIG. 7B shows a flowchart explaining the operation of the virtual IP Converter with respect to keyboard and mouse controls.

### DESCRIPTION OF THE EMBODIMENTS

Referring now to the drawings, wherein like reference numerals designate identical or corresponding parts throughout the several views.

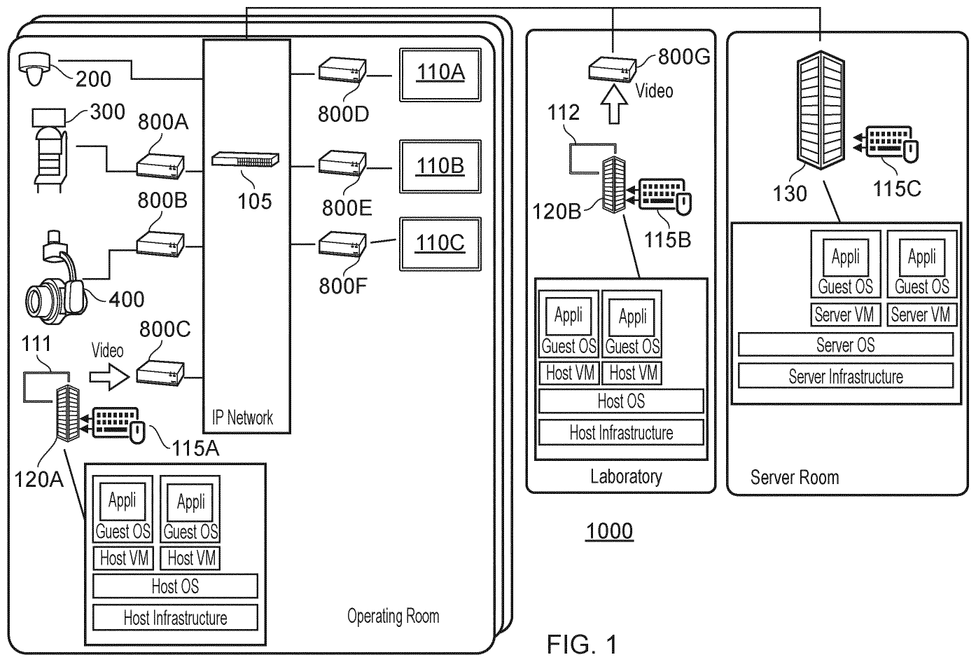

FIG. 1 is a schematic diagram illustrating a medical system 1000 connected to a network. The medical system 1000 is provided between one or more operating rooms, a laboratory and a server room. In embodiments, the network is an Internet Protocol (IP) network. Of course, the disclosure is not so limited and any kind of network is envisaged.

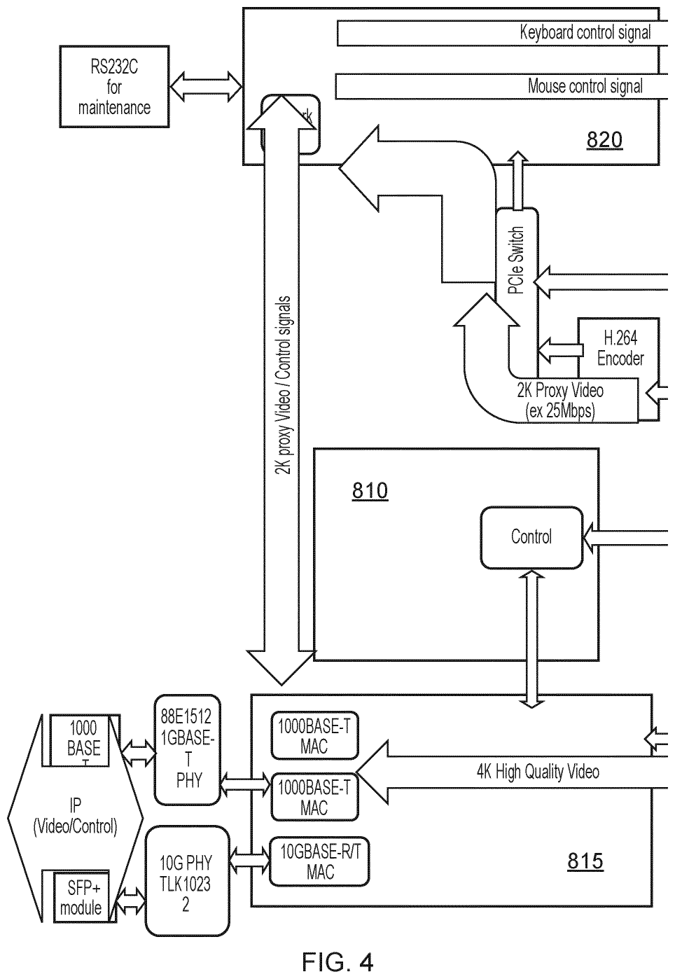

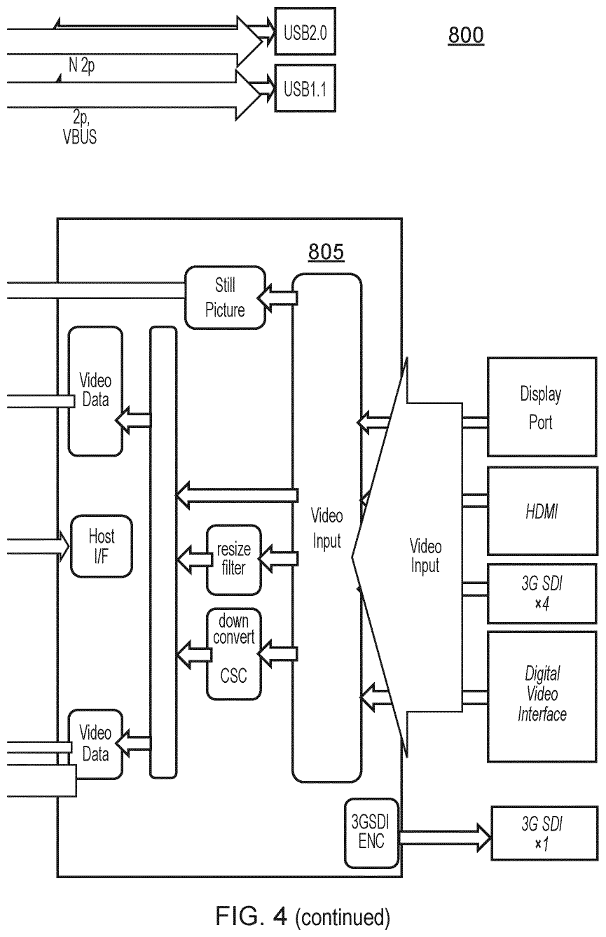

The medical system 1000 includes a server 130 (located in the server room), an operating room camera 200, an endoscope system 300, a room camera 400, an Operating Room (OR) workstation 120A having a keyboard and mouse 115A, a laboratory workstation 120B having a monitor 112 and keyboard and mouse 115B and a monitor 111 connected thereto and viewing monitors 110A, 110B, 110C which allows the surgical team to view the progress of the surgery. In embodiments, the endoscope system 300, the room camera 400, the OR workstation 120A, the laboratory workstation 120B and each of the viewing monitors 110A-C is each connected to the receiving side of the physical IP Converter 800D-F (IPC-RX). These devices except the laboratory workstation 120B are all located in the operating room. Each IP Converter 800A-C, G (IPC-TX) takes the image data (such as video data from the OR workstation 120A, the laboratory workstation 120B, a room camera 400 or the endoscope system 300), control command data and status data etc produced by each device and converts that data into IP packets when providing IP packets onto the IP network. An explanation of the functionality of the transmission side of the IP Converter 800A-C, G (IPC-TX) when converting data into IP packets will be described later with reference to FIG. 4. Conversely, each IP converter 800D-F (IPC-RX) receives IP packets from the IP network and outputs image data, control command data and status data to the device to which the IP converter 800D-F (IPC-RX) is attached. The explanation of the functionality of the receiving side of the IP Converter is omitted as this is not the focus point of this disclosure.

After conversion into IP packets, those IP packets are then fed into a network switch 105 also located in the operating room which routes the IP packets to the appropriate device in the operating room or onto the IP network. Although it is not described in the figure, network switch may be located outside of the operating room, for example in the server room. As will be evident, the operating room camera 200 is not connected to transmission side of an IP Converter as the output from the operating room camera 200 is IP packets. Accordingly, the operating room camera 200 is directly connected to the network switch 105.

In the laboratory, a laboratory workstation 120B having a monitor 112 and keyboard and mouse 115B connected thereto is provided. Again, the laboratory workstation 120B produces a video stream output and this is fed into an IP Converter 800G (IPC-TX) connected to the IP network. The IP converter 800G converts the video stream into IP packets for provision over the IP network, where IP packets are routed appropriately.

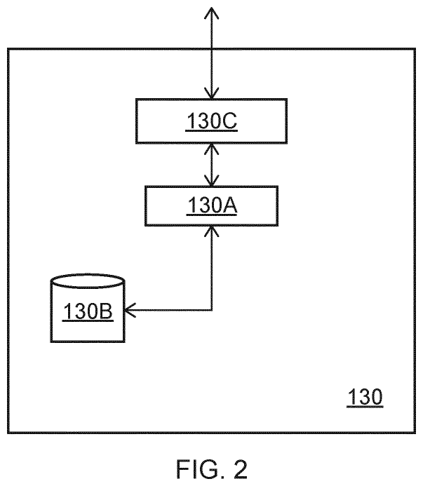

In the server room a server 130 is provided. The server 130 comprises server processor circuitry 130A and server storage 130B. The server 130 is connected to the IP Network using server network circuitry 130C. The server 130 is shown in FIG. 2.

Referring back to FIG. 1, the OR workstation 120A includes a first application and a second application. These applications are computer programs or groups of programs which are designed for end users and which perform specific tasks. In the context of an OR workstation 120A, one such application allows multiple video feeds to be processed and output to the different viewing monitors 110A-C in the Operating Room via IP Converter 800C (IPC-TX), network switch 105, and the IP converter 800D-F (IPC-RX). For case of explanation, it will be assumed that the first application has a video data output produced. Of course, the disclosure is not so limited and the second application may also or instead produce a video data output.

The first application and/or second application are provided on a Guest Operating System (this is referred to as a “Guest OS” in FIG. 1) such as Windows R, Linux etc. The Guest Operating System is the Operating System on which the first application and/or second application operates. In other words, the Guest Operating System is software installed and run on the Virtual Machine (the “Host VM” in FIG. 1). The Host VM is run on the Host Operating System which is the Operating System of the physical OR workstation 120A. This may be the same as or different to the Guest Operating System (which may be Windows R, Linux, etc or may be a different version of the Guest Operating System).

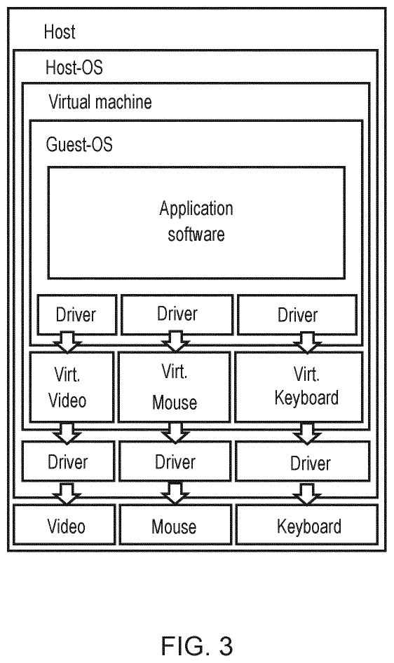

Additionally, as noted above, connected to the OR workstation 120A is the physical keyboard and mouse 115A. As will be appreciated, this physical keyboard and mouse 115A will need drivers for the Host Operating System to function correctly. Moreover, as noted above, video data produced by either the first application and/or second application needs to be provided to the monitor 111 and to the IP network via IP Converter 800C (IPC-TX). This means that video drivers for the Host Operating System will also be required. This will be explained later with reference to FIG. 3.

In case of both the first and the second applications in the OR workstation 120A having video outputs, the Host Operating System typically uses a virtual desktop, in which both applications are displayed simultaneously in individual small size of a child screen out of full size screen.

The skilled person will appreciate that the server 130 of FIG. 1 has a known setup. Specifically, the applications loaded onto the server storage 130C are to be run on the Guest Operating System. The Guest Operating System is to be run on a virtual machine in the workstations and so the Guest Operating System within the server 130 sits on a corresponding server virtual machine. The server virtual machine sits on a server Operating System which itself sits on the physical server infrastructure. Similarly, the skilled person will prepare the OR workstation 120A and the laboratory workstation 120B with virtualization.

In a typical medical procedure, the endoscope system 300 is used by a surgeon to perform the medical procedure. The output from the endoscope system 300 is typically video showing the area being investigated by the surgeon. This video is converted into IP packets in the IP converter 800A (IPC-TX). Those IP packets are provided to the network switch 105 and are fed to a viewing monitor 110A-C for display to the surgical team. In order to be displayed on the viewing monitor 110A-C, the IP packets are routed, by the network switch 105 to an IP converter 800D-F (IPC-RX) attached to the correct viewing monitor 110A-C. The IP converter 800D-F (IPC-RX) converts the IP packets transferred by the network switch 105 into a video signal and this is displayed on the viewing monitor 110A-C.

In other scenarios, the video output by the endoscope system 300 is converted into IP packets by the IP converter 800A (IPC-TX) and fed into the network switch 105. Instead of being routed directly to the viewing monitor 110A-C, the IP packets are fed to the server 130, where the IP stream might be recorded, edited, broadcast etc. In some medical applications, multiple streams of video data, such as video data captured of the area being investigated using various wavelengths of light, are overlaid to produce a composite image which is useful to the surgeon in performing his or her investigation. Namely, the various kinds of video data are used through hospital IP network not only during the surgery such as viewing, broadcasting etc, but also after the surgery such as recording, editing etc.

In FIG. 3 corresponding to the OR workstation 120A and the laboratory workstation 120B in FIG. 1, one of the applications is running on the Guest Operating System. For simplicity, the OR workstation 120A is described here. As noted above, this will be explained with reference to the first application. The first application running on the Guest Operating System requires a keyboard and mouse input (for control) and will produce video data as an output. Accordingly, a Guest Operating System driver for the keyboard, mouse and video will need to be provided. The first application is running on the Guest Operating System (which may be the same as or different to the Host Operating System), which itself is running on a virtual machine within the OR workstation 120A. Therefore, to allow the Host Operating System to process the instructions from the physical keyboard and mouse 115A to the virtual machine, and to provide the video produced in the virtual machine to be output to the physical monitor 111, a set of Host Operating System drivers is required. This means that in order to allow a virtual machine running the first application to operate on the OR workstation 120A, the first application may need adapting so that the Host Operating System drivers may operate correctly. Alternatively, the Host Operating System drivers may need adapting.

Referring to FIG. 4, the transmission side of an IP Converter 800A-C, G (IPC-TX) is shown. As noted above, the transmission side of IP Converter 800 A-C, G (IPC-TX) is known hardware that converts video and other image data, control and status data and mouse and keyboard controls into IP packets which are then provided to the IP network. These IP packets may be routed via network switch 105 to the IP packet's final destination. It will be appreciated that in the known receiving side of IP Converter 800D-F (IPC-RX), IP packets received at the IP Converter 800 would be converted to video and other control signals. However, this reception of IP Packets is not described for brevity.

The transmission side of IP converter 800 A-C, G (IPC-TX) comprises one video input from various inputs. In the example of FIG. 4, the IP Converter 800 A-C, G (IPC-TX) has a display port input, a High Definition Multimedia Interface (HDMI) input, 4×3G Serial Digital Interface (SDI) inputs, and a Digital Visual Interface (DVI) input. Obviously, the IP Converter 800A-C, G (IPC-TX) may comprise different video and image connectors and such selection and number is limited by the physical size and configuration of the IP Converter 800A-C, G (IPC-TX).

The one video input is fed into video processing circuitry 805 which generates two output video streams. The first video stream is a high definition video stream. This high definition video stream may be at 4K or 8K resolution, for example. The second video stream is a lower resolution version of the high definition video stream. In the example of FIG. 4, the second video stream is at 2K resolution and is termed a proxy video. That is, the second video stream is a proxy version of the first video stream. The operation of the video processing circuitry 805 is known and so will not be explained in detail for brevity.

The first video stream is output to communication circuitry 815. The communication circuitry 815 converts the first video stream into IP packets for transmission over the IP network.

The second video stream is output to control circuitry 820. The control circuitry 820 processes the second video stream and provides the processed second video stream to the communication circuitry 815. In addition to the second video stream, control data for the second video stream is also provided to the communication circuitry 815. For example, control data for any production of the second video stream (such as H.264 Encoding control data) is also provided to the communication circuitry 815. The communication circuitry 815 converts the second video stream and the control data for the second video stream into IP packets which are sent over the IP network.

The control circuitry 820 also provides control data to and receives control data from the keyboard and mouse which are connected to USB connectors.

The operation of transmission side of the IP converter 800 A-C, G (IPC-TX) is controlled by processing circuitry 810 that is connected to the video processing circuitry 805 and the communication circuitry 815.

As noted above, the transmission side of an IP Converter 800A-C, G (IPC-TX) is known.

However, there has been an increased use of software in the medical environment which aims to remove as much hardware as possible from the hospital system to reduce the space. Therefore, virtualizing the applications stored on and used by the OR workstation 120A and the laboratory workstation 120B is desirable. Here, as an example, the first application running on the OR workstation 120A working on the Guest Operating System, which uses keyboard and mouse input (for control) and produces video data as an output is virtualized on server 130 in order to reduce the requirement to have physical OR workstation 120A. Likewise the first application running on the laboratory workstation 120B working on the Guest Operating System, which uses keyboard and mouse input (for control) and produces video data as an output is virtualized on server 130 in order to reduce the requirement to have physical laboratory workstation 120B server. Additionally, individual IP converter 800C, 800G (IPC-TX) to output the video as IP packets onto the IP network on each workstation should be desirable to be removed in conjunction with the virtualization and the reduction of keyboard and mouse should be also considered.

However, this virtualisation is not easily possible. There are 4 concerns to be solved when virtualisation of medical software is adopted.

Firstly, as such software previously run on the local workstations usually includes video processing which output multiple video streams, it is complicated to virtualize such software as additional hardware is required at the virtual server to provide multiple video outputs. Additionally, in a hospital application, the location and the numbers of keyboard/mouse are a concern. End users want to control keyboard/mouse from the other sites in the hospital remotely, and to reduce the number of keyboard/mouse in order to save space and cost. The first concern is how to treat a keyboard, video stream and mouse when the virtualisation is adopted.

Secondly, less porting effort for virtualisation is expected. When the software worked on standalone local workstation is virtualised on large servers located in a server room, some modifications are practically required such as memory control, disk control, etc in addition to the KVM control, as the software is basically designed for specific hardware, operating system, interface and so on. This modification generally requires development efforts (human resource, development environment, cost, time etc). Moreover, it indirectly connects to the reduction of quality or reliability. Further, it causes the additional maintenance cost after the production launch caused by the modification from the original software worked on standalone local workstation. No modification is desired to avoid these concerns.

Thirdly, similarly to the second concern, minimization of the impact to the existing other software is desirable. When the software worked on standalone local workstation is virtualised on large servers located in a server room, the impact to other software not only working on virtualised workstation but also working on other areas should be minimized or at least reduced.

Lastly, software run on local workstations is often classified as a medical device and is subjected to regulatory approval. Therefore, by simply placing the software on a virtual server will mean that all applications within the virtual server (including its operating system) would need the same regulatory approval to operate by virtue of the presence of the software previously run on the local workstations. When the software worked on standalone local workstation is virtualised on large servers located in a server room, the clear separation of medical device and non-medical device is essential for all software used in the hospital in terms of intended use of the software and impact analysis to the effectiveness and safety for medical usage.

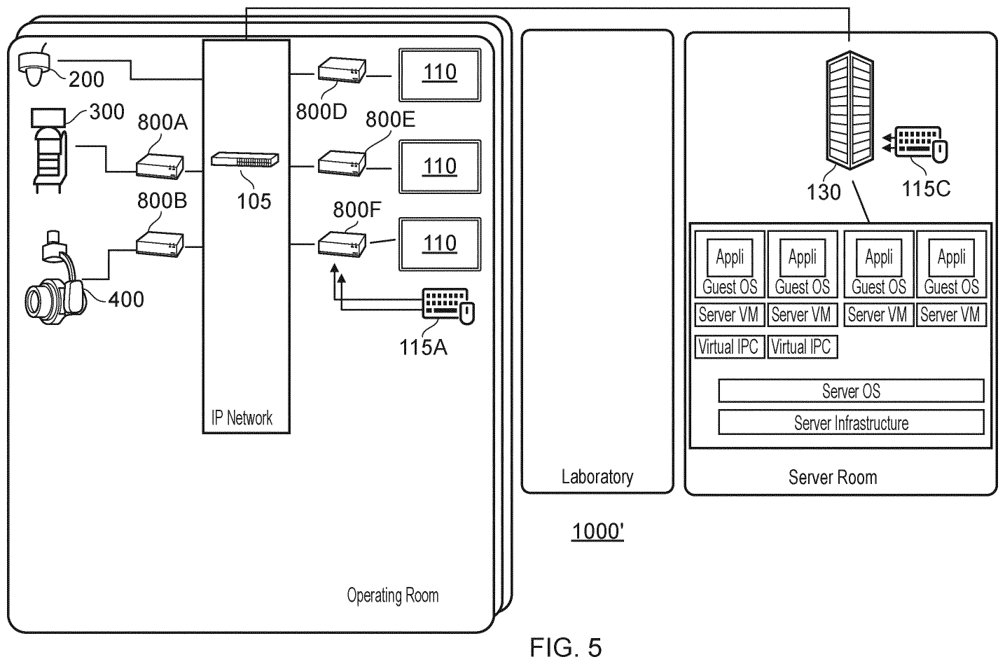

FIG. 5 shows a system 1000′ according to embodiments of the disclosure. These embodiments mean that a virtual IP Converter is adopted to address at least one of the above concerns. In instances, the virtual IP converter addresses the four concerns at the same time. The first application that was run on the OR workstation 120A in FIG. 1 has, in the embodiments of FIG. 5, been installed in, and operated from, the server 130 located in the server room. In other words, the first application has been virtualised by being run from the server 130. In particular, the server 130 is a virtual server and that virtual server is stored in storage 130B. Although the second application on the OR workstation 120A is not described explicitly in FIG. 5, analogously, the first application on the OR workstation 120A is adopted. The first application that was run on the laboratory workstation 120B in FIG. 1 has in the embodiments of FIG. 5 been installed in, and operated from the server 130 located in the server room. In other words, the first application has been virtualised by being run from the server 130. In particular, the server 130 is a virtual server and that virtual server is stored in storage 130B. Although the second application on the OR workstation 120B is not described explicitly in FIG. 5, analogously, the first application on the OR workstation 120B is adopted.

In the system of FIG. 5, the physical OR workstation 120A of FIG. 1 and the physical laboratory Workstation 120B of FIG. 1 have been removed. This creates more space in the operating room and the laboratory and reduces the number of items in the respective rooms that need cleaning. Instead, the keyboard and mouse 115A are connected into the USB connectors in one of the IP Converters 800F (IPC-RX) located in the operating room. As explained with reference to FIG. 4, this means that the control signals from the keyboard and mouse are transferred over the IP network as IP packets. The keyboard and mouse 115A enable to control the first application that was run on the OR workstation 120A in FIG. 1 and now run on the server 130 and the first application that was run on the laboratory workstation 120B in FIG. 1 and now run on the server 130 exclusively. The reduction of the number of keyboard and mouse is achieved in contrast to FIG. 1. Additionally. IP Converter 800C and 800G in FIG. 1 are also removed.

The first applications virtualized from the OR workstation 120A of FIG. 1 and the Laboratory Workstation 120B of FIG. 1 are provided on the Guest Operating System within the server storage 130B. There is a driver located within the first application that that allows the first application to output video data and be controlled using the keyboard and mouse within the virtual machine located on the server (termed the Server VM in FIG. 5). However, instead of the virtual machine in the server 130 being provided directly on a Server Operating System that would require the provision of a different set of drivers as in the system of FIG. 1, the first applications virtualized from the OR workstation 120A of FIG. 1 and the Laboratory Workstation 120B of FIG. 1 is provided on a virtual IP Converter according to embodiments of the disclosure.

The purpose of the virtual IP Converter is to convert the video output from the first application on the virtual machine directly into IP packets. As will be explained later, the virtual IP converter will be seen by the first application running on the virtual machine as a video connector like those described in the IP Convertor 800 of FIG. 4. This means that no additional driver is required to output the video data and no modification of the first application is required.

Moreover, the keyboard and mouse control signals being provided to the first application will be provided as IP packets which will then be output to the virtual machine as control signals complying with the USB interface.

In other words, by providing the virtual IP converter beneath the virtual machine running the first application, the Guest Operating System running on the virtual machine will see the video data, the keyboard and the mouse as standard hardware. This means that no additional driver is required and so no modification of the first application is required. This allows virtualisation of the first application onto the server 130 and the removal of the OR workstation 120A and the laboratory workstation 120B.

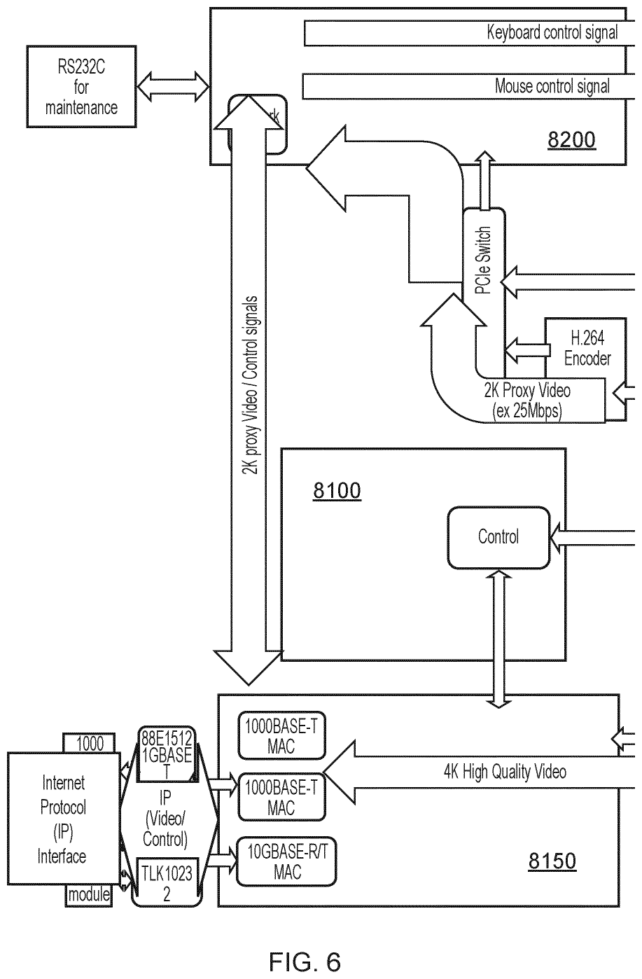

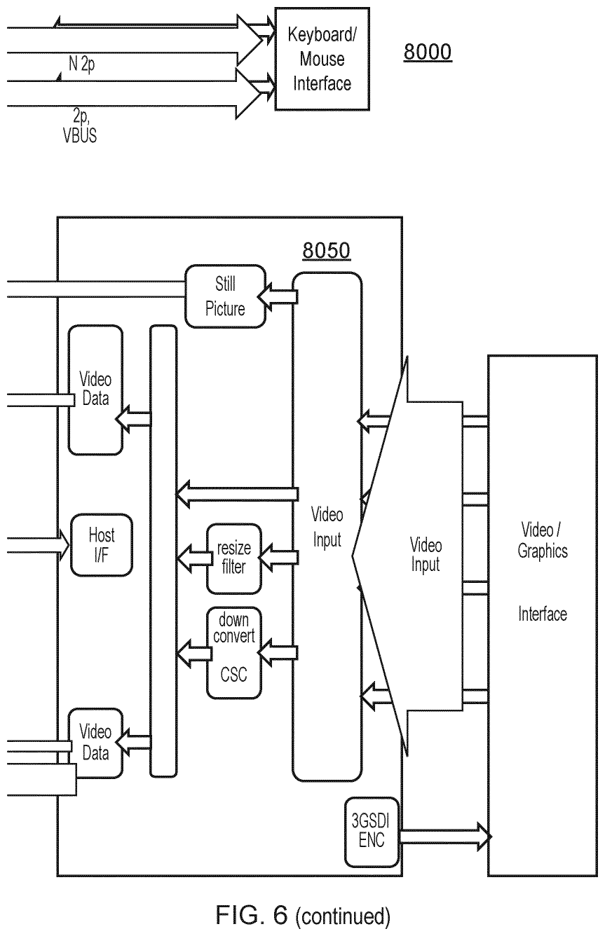

FIG. 6 shows the transmission side of a virtual IP converter 8000 according the embodiments of the disclosure. As described above, the virtual IP converter 8000 sits beneath the virtual machine upon which the first application is running and replicates the operation of the transmission side of IP converter 800 (IPC-TX) of FIG. 4. In particular, the virtual IP converter 8000 is software code that is stored on the server storage 130B and is designed to replicate the operation of the transmission side of the IP converter 800 (IPC-TX) of FIG. 4. In embodiments, the virtual IP converter is referred to as an “IP converter application”.

The video data output by the first application is provided to the video graphics interface. The format of the video data output may be HDMI, DVI, SDI or the like. Moreover, although the physical number and types of ports within the IP converter 800 (IPC-TX) is limited, in the virtual IP converter 8000 no such limit exists as the video interface is run in software located within the server 130. Specifically, the video interface is a function call for video data from the first application.

The video data from the video interface is fed into virtual video processing code 8050 which is run as part of the virtual IP converter 8000. The virtual video processing code 8050 performs a similar function to the video processing circuitry 805 described with reference to FIG. 4. Specifically, the one or more video or image inputs from the video interface are fed into the virtual video processing code 8050 which generates two output video streams. The first video stream is a high definition video stream. This high definition video stream may be at 4K or 8K resolution, for example. The second video stream is a lower resolution version of the high definition video stream. In the example of FIG. 6, the second video stream is at 2K resolution and is termed a proxy video. That is, the second video stream is a proxy version of the first video stream.

The first video stream is output to virtual communication code 8150. The virtual communication code 8150 converts the first video stream into IP packets for transmission over the IP network.

The second video stream is output to virtual control code 8200. The virtual control code 8200 processes the second video stream and provides the processed second video stream to the virtual communication code 8150. In addition to the second video stream, control data for the second video stream is also provided to the virtual communication code 8150. For example, control data for any production of the second video stream is also provided to the virtual communication code 8150. The virtual communication code 8150 converts the second video stream and the control data for the second video stream into IP packets which are sent over the IP network. The video data relates to a medical procedure.

In embodiments of the disclosure, the second video stream may be recorded, edited or broadcast. In other words, the virtual IP converter 8000 may record, edit or broadcast the second output video stream as IP packets over the IP network.

For example, the second video stream may be used for editing. It is advantageous to use a lower resolution video stream for editing as less processing of the video is required. The edited lower resolution video stream may produce an edit decision list which can be applied to the high definition video stream to produce edited high resolution content. In instances, the lower resolution video of the second stream may be stored as a record of the surgery. This record may be used for training purposes for surgical students or may be used to confirm that the surgeon has performed the surgery correctly. By storing the lower resolution video stream, less storage is required. Similarly, the lower resolution second video may be used to broadcast the surgery to students who may be located remotely from the OR. This ensures that bandwidth of the network is used efficiently.

The virtual control code 8200 also provides control data to the virtual keyboard and mouse which are used to control the keyboard and mouse in the virtual machine upon which the Guest Operating System and the first application are run.

In addition to converting the video stream into IP packets and providing these IP packets onto the IP network, the IP converter application is configured to receive keyboard and/or mouse instructions as IP packets over the IP network. These keyboard and/or mouse instructions are received from the keyboard and/or mouse 115A being inserted into the IP converter 800F (IPC-RX) in the operating room. These IP packets are converted into keyboard and/or mouse control signals in a format, the converted keyboard and/or mouse control signals being passed to the medical application within the virtual server. In particular, the surgical team uses the keyboard and/or mouse 115A to control the medical application on the virtual server. In order to achieve this, the keyboard and/or mouse control signals are converted into IP packets within the receiving side of IP converter 800F (IPC-RX) for transmission over the IP network.

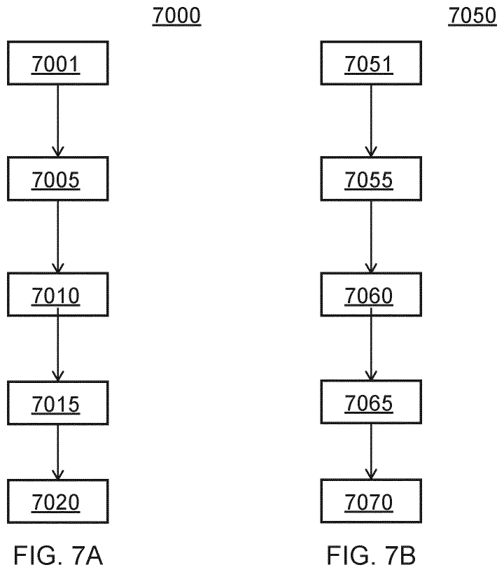

FIG. 7A shows a flowchart 7000 explaining the operation of the transmission side of the virtual IP Converter 8000 with respect to video. The process starts at step 7001. The process moves to step 7005 where the video data from the first application is received at the video interface. The process moves to step 7010 where the video is processed, by being, for example, converted into the first video stream and the second video stream. The process moves to step 7015 where the video is converted into IP packets which are sent over the IP network. The process ends at step 7020.

FIG. 7B shows a flowchart 7050 explaining the operation of the virtual IP Converter 8000 with respect to keyboard and mouse controls. The process starts at 7051. The process moves to step 7055 where the IP packets are received by the virtual communication code 8150. The process moves to step 7060 where the IP packets are converted in the virtual communication code 8150 into control signals for the keyboard and mouse. Specifically, the virtual communication code 8150 converts the IP packets into control data conforming to the USB standards. The process then moves to step 7065 where the control data is sent to the keyboard and mouse interface in the virtual machine which is used by the Guest Operating System to control the first application. The process ends in step 7070.

Although the foregoing has been described with reference to the mouse and/or keyboard being compliant with a Universal Serial Bus Standard such as USB, the disclosure is not so limited. For example, the mouse and/or keyboard may be compliant with PS/2 standard or the like. Moreover, the disclosure is not limited to keyboard and mouse and any control mechanism for the first application such as a touch screen control data is envisaged. Additionally, although the Figures describe the mouse and keyboard being used, either of the keyboard or the mouse may be used on its own. Therefore, keyboard and mouse should be interpreted as keyboard and/or mouse.

According to embodiments, the described virtual IP Converter, addresses one or more of the four concerns noted above when virtualisation of medical software is adopted. Firstly, the virtual IP Converter enables video processing which output multiple video streams as well as the keyboard/mouse control. The virtual IP Converter according to embodiments can absorb the difference of the platform, so less porting effort for virtualisation of existing application, and the minimization of the impact to the existing other software is achieved. Lastly, the virtual IP Converter according to embodiments keeps the isolation of each software modules so that the boundary of medical device and non-medical device is kept intact.

As the virtual IP Converter outputs IP packets to an IP network, it is treated just as one of the video source signals like operating room camera 200 in FIG. 1 directly connected to the network switch 105. No special customizations are therefore required. This effect also contributes to the simplicity to the whole hospital systems.

Although the foregoing has been described with reference to a medical procedure, the disclosure is not so limited and may be implemented in any situation where data is passed around an IP network.

Numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed, the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

Embodiments of the present technique can generally described by the following numbered clauses:

1. A virtual server configured to receive and transfer data over an Internet Protocol (IP) network, the virtual server comprising:
a medical application configured to process video data relating to a medical procedure to produce an output video stream, the medical application being provided on a Guest Operating System within the virtual server; and
an IP converter application sat beneath the Guest Operating System configured to receive the output video stream from the medical application and to convert the at least one video stream into IP packets and to transfer the converted video data over the IP network.

2. A virtual server according to clause 1, wherein the IP converter application is configured to receive keyboard and/or mouse instructions as IP packets over the IP network, and to convert the received keyboard and/or mouse instructions from IP packets into keyboard and/or mouse control signals in a format, the converted keyboard and/or mouse control signals being passed to the medical application.

3. A virtual server according to any preceding clause, wherein the IP converter application is configured to transfer a second output video stream from the medical application, wherein the second output video stream is a lower resolution version of the output video stream.

4 A virtual server according to clause 3, wherein the IP converter application is configured to record, edit or broadcast the second output video stream as IP packets over the IP network.

5. A virtual server according to any preceding clause, wherein the data relates to a medical procedure.

6. A medical system comprising a monitor configured to receive video data as IP packets; and a virtual server according to any preceding clause.

7. A method of operating a virtual server configured to receive and transfer data over an Internet Protocol (IP) network, the method comprising:

processing video data in a medical application relating to a medical procedure to produce an output video stream, the medical application being provided on a Guest Operating System within the virtual server; and providing an IP converter application beneath the Guest Operating System, the IP application receiving the output video stream from the medical application and converting the at least one video stream into IP packets and transferring the converted video data over the IP network.

8. A method according to clause 7, wherein the IP converter application receives keyboard and/or mouse instructions as IP packets over the IP network, and converts the received keyboard and/or mouse instructions from IP packets into keyboard and/or mouse control signals in a format, the converted keyboard and/or mouse control signals being passed to the medical application.

9. A virtual server according to either one of clause 7 or 8, wherein the IP converter application transfers a second output video stream from the medical application, wherein the second output video stream is a lower resolution version of the output video stream.

10. A virtual server according to any one of clauses 7 to 9, wherein the data relates to a medical procedure.

11. A computer program product comprising computer readable instructions which, when loaded onto a computer, configures the computer to perform a method according to any one of clauses 7 to 10.

1. A virtual server configured to receive and transfer data over an Internet Protocol (IP) network, the virtual server comprising:
a medical application configured to process video data relating to a medical procedure to produce an output video stream, the medical application being provided on a Guest Operating System within the virtual server; and
an IP converter application sat beneath the Guest Operating System configured to receive the output video stream from the medical application and to convert the at least one video stream into IP packets and to transfer the converted video data over the IP network.

2. A virtual server according to claim 1, wherein the IP converter application is configured to receive keyboard and/or mouse instructions as IP packets over the IP network, and to convert the received keyboard and/or mouse instructions from IP packets into keyboard and/or mouse control signals in a format, the converted keyboard and/or mouse control signals being passed to the medical application.

3. A virtual server according to claim 1, wherein the IP converter application is configured to transfer a second output video stream from the medical application, wherein the second output video stream is a lower resolution version of the output video stream.

4. A virtual server according to claim 3, wherein the IP converter application is configured to record, edit or broadcast the second output video stream as IP packets over the IP network.

5. A virtual server according to claim 1, wherein the data relates to a medical procedure.

6. A medical system comprising a monitor configured to receive video data as IP packets; and a virtual server according to claim 1.

7. A method of operating a virtual server configured to receive and transfer data over an Internet Protocol (IP) network, the method comprising:
processing video data in a medical application relating to a medical procedure to produce an output video stream, the medical application being provided on a Guest Operating System within the virtual server; and
providing an IP converter application beneath the Guest Operating System, the IP application receiving the output video stream from the medical application and converting the at least one video stream into IP packets and transferring the converted video data over the IP network.

8. A method according to claim 7, wherein the IP converter application receives keyboard and/or mouse instructions as IP packets over the IP network, and converts the received keyboard and/or mouse instructions from IP packets into keyboard and/or mouse control signals in a format, the converted keyboard and/or mouse control signals being passed to the medical application.

9. A virtual server according to claim 7, wherein the IP converter application transfers a second output video stream from the medical application, wherein the second output video stream is a lower resolution version of the output video stream.

10. A virtual server according to claim 7, wherein the data relates to a medical procedure.

11. A computer program product comprising computer readable instructions which, when loaded onto a computer, configures the computer to perform a method according to claim 7.
